(19) United States
(12) Patent Application Publication
(10) Pub. No.: US 2025/0339314 A1
(43) Pub. Date: Nov. 6, 2025

(54) Ocular Chemical Injury Treatment Ring and Method of Use

(71) Applicant: The Government of the United States, as Represented by the Secretary of the Army, Fort Detrick, MD (US)

(72) Inventor: Gregory Bryant Giles, Hopkinsville, KY (US)

(21) Appl. No.: 18/854,087

(22) PCT Filed: Apr. 20, 2023
(86) PCT No.: PCT/US2023/019172

§ 371 Date: Oct. 4, 2024

Related U.S. Application Data
(60) Provisional application No. 63/333,146, filed on Apr. 21, 2022.

(51) Int. Cl. A61F 9/00; A61M 3/02

(52) U.S. Cl. CPC A61F 9/0017; A61M 3/0258; A61M 3/0279; A61M 2205/3324; A61M 2210/0612

(57) ABSTRACT

An ocular chemical injury treatment ring comprises a substantially circular ring of a medical grade material and having a plurality of fenestrations or holes around a circumference of the circular ring.

This PCT international application claims priority to U.S. Ser. No. 63/333,146 filed on 21 Apr. 2022 in the U.S. Patent and Trademark Office, the entirety of which is incorporated herein by reference.

### FIELD OF INVENTION

The present invention is directed to an ocular chemical injury treatment ring and to methods of use thereof. In particular, the ocular chemical injury treatment ring is a fenestrated ring that is configured to fit into the fornices of the eye and may be used to treat chemical injuries to the eye.

### BACKGROUND OF INVENTION

Chemical injuries to the eye are one of the relatively few sight-threatening ocular emergencies. Chemical injuries represent up to 22% of ocular trauma. As chemicals come in contact with the eye, damage begins to occur immediately. Acidic chemicals begin a process of tissue coagulation, while alkali chemicals lead to saponification. Alkali injuries are particularly damaging as the initial damage leaves tissue more susceptible to further penetration.

Factors that influence the degree of damage include one or more of chemical pH, amount of chemical entering the eye, presence of particulate matter, time to neutralization, or any remaining chemical and/or particulate matter in the fornices. Chemical injuries can range anywhere on the pH spectrum. The further away from the normal eye pH, the more caustic the chemical injury. Irreversible damage may occur at a pH above 11.5.

The recommended initial treatment for eye injuries is copious liquid irrigation to remove residual chemicals and/or particulates and restore the physiologic pH to prevent further damage. The most common irrigation methods include manual irrigation, trickle/drip, and the Morgan MEDI-FLOW® Lens.

Manual liquid irrigation includes flushing one or both eyes by a hand-held method (e.g., via a bottle or syringe) or by a stationary method (e.g., an eye flush station). These methods are generally used for initial wash-out at the time of injury; however, it is difficult to sustain for an extended period of time and may require more than one person to accomplish.

A trickle/drip liquid irrigation involves a low flow over the eye while a patient is asked to blink or keep the eyes open. This procedure allows a continuous flow; however, it is dependent on patient cooperation. As fluid drips over the eye, irrigation is not directed under the eyelids. Irrigation relies on patient blinking to force fluid into the fornix, or the deep pocket under the eyelids that surrounds the eye. An eyelid speculum may be required to keep the eye open.

The most commonly used method for irrigation is via the Morgan MEDI-FLOW® Lens. This lens in inserted under the eyelids, vaults over the clear cornea, and allows for a continuous, passive flow of fluid around the lens and under the palpebral conjunctiva. The lens is typically attached to an intravenous bag and can be pressurized for faster irrigation. However, this lens may not adequately irrigate the fornices.

Following these types of irrigation, the pH is periodically checked and the fornices are swept/cleansed to remove any retained particulate matter. This checking and cleansing are typically performed by an emergency department physician or an ophthalmologist and may not be performed until hours after the initial injury. Therefore, irrigation may not be as effective because particulate matter under deeper fornix tissue may continue to produce chemical or other injury.

Accurate pH testing is important to guide treatment. Failure to accurately assess pH in the fornices can lead to sustained ocular damage. Acute injuries may lead to corneal edema and/or opacification, ocular ulceration/perforation, and elevated intraocular pressure as tissue is damaged. Long term effects include, but are not limited to, one or more of corneal haze, corneal scarring, neovascularization, glaucoma, decreased vision, or blindness. Ocular pH is often tested with litmus paper. Litmus paper pH testing is quick and easy to perform, although several limitations exist. Litmus paper does not provide a quantitative result. Often, litmus strips are light exposed and/or degraded, leading to inaccurate pH measurements. In unilateral chemical injuries, one eye can be tested against the fellow eye. However, in bilateral cases a third eye may be needed as a reference. The litmus paper strips cannot be placed deep in the fornix, so the recorded pH of the cornea may not be indicative of true pH in the more dependent areas of the eye, such as the fornices.

### SUMMARY OF INVENTION

The invention provides in a first embodiment an ocular chemical injury treatment ring comprising a substantially circular ring of a medical grade material and having a plurality of fenestrations or holes around a circumference of the circular ring. The circular ring may be made of intravenous tubing or plastic.

The invention provides in a second embodiment further to any of the previous embodiments an ocular chemical injury treatment ring having fenestrations equally spaced about the circumference of the circular ring. The fenestrations may be of the same size or a different size. There may be 20 to 50 fenestrations around the circumference of the circular ring.

The invention provides in a third embodiment further to any of the previous embodiments an ocular chemical injury treatment ring in which each of the plurality of fenestrations is angled with respect to a central longitudinal axis of the circular ring. Each fenestration may comprise a V-shaped opening or hole.

The invention provides in a fourth embodiment further to any of the previous embodiments an ocular chemical injury treatment ring in which each of the fenestrations comprises an opening of about 0.1 mm to about 0.5 mm.

The invention provides in a fifth embodiment further to any of the previous embodiments an ocular chemical injury treatment ring in which the plurality of fenestrations comprises a first set of holes having a first size and a second set of holes having a second size larger than the first size.

The invention provides in a sixth embodiment further to any of the previous embodiments an ocular chemical injury treatment ring having at least two sets of holes, in which each set of holes is positioned at a different location around a circumference of the circular ring and around a circumference of tubing forming the circular ring.

The invention provides in a seventh embodiment further to any of the previous embodiments an ocular chemical injury treatment ring, wherein the substantially circular ring, at a proximal end, is integral with a section of tubing having no fenestrations and having a connector configured to attach to a pump or intravenous bag.

The invention provides in an eight embodiment further to any of the previous embodiments an ocular chemical injury treatment ring having no corneal shield or dome.

The invention provides in a first method embodiment a method for treating an eye comprising inserting an ocular chemical injury treatment ring according to any of the previous embodiments into an eye, such that the circumference of the ring is at least partially beneath an eyelid and substantially in or adjacent to the fornices; inserting or pumping a liquid into the circular ring and out of the plurality of fenestrations at a flow rate that creates a circular and/or vortex pattern in the fornices; and irrigating the fornices and cornea. At least one of chemicals or particulate matter is removed from the fornices.

The invention provides in a first kit embodiment a kit comprising a housing; at least one ocular injury treatment ring according to any of the previous embodiments; at least one pump; and a rechargeable battery. The kit may also have a pH meter comprising a sterilizable unitary body having a single point configured to measure the pH of the eye.

An advantage of the ocular chemical injury treatment ring of present invention is that it is easily insertable into the fornices of the eye.

Another advantage of the ocular chemical injury treatment ring of the present invention is that the plurality of fenestrations actively irrigates the fornices, where chemicals and/or particulate matter may be difficult to remove.

### BRIEF DESCRIPTION OF THE DRAWINGS

FIG. 1A is a top view of an ocular chemical injury treatment ring according to an embodiment of the present invention.

FIG. 1B is a top view of an ocular chemical injury treatment ring with angled fenestrations according to an embodiment of the present invention.

FIG. 1C is a top view of an ocular chemical injury treatment ring according to an embodiment of the present invention, with a different orientation of fenestrations than in FIGS. 1A-1B.

FIG. 1D is a top view of an ocular chemical injury treatment ring with a T-junction or connector according to an embodiment of the present invention.

FIG. 2 is a perspective view of an ocular chemical injury treatment ring with a connector.

FIG. 3 is a schematic view of an ocular chemical injury treatment ring with corneal shield and struts according to another embodiment of the present invention.

FIG. 4 is a perspective view of a kit according to an embodiment of the present invention.

### DETAILED DESCRIPTION OF THE INVENTION

The present invention is directed to an ocular chemical injury treatment ring and methods of use thereof. In particular, the ocular chemical injury treatment ring is a fenestrated ring that is configured to fit into the fornices of the eye and may be used to treat chemical injuries to the eye. Although the use of the ocular treatment ring to treat chemical injury to the eye is discussed below, it is readily apparent that the ocular treatment ring can be used to treat other injuries that require eye irrigation and may also be used to administer medicine to an eye, with a selectable range of liquid speed and quantity.

In this detailed description, references to “one embodiment”, “an embodiment”, or “in embodiments” mean that the feature being referred to is included in at least one embodiment of the invention. Moreover, separate references to “one embodiment”, “an embodiment”, or “embodiments” do not necessarily refer to the same embodiment; however, neither are such embodiments mutually exclusive, unless so stated, and except as will be readily apparent to those skilled in the art. Thus, the invention can include any variety of combinations and/or integrations of the embodiments described herein.

As used herein “substantially”, “generally”, “about”, and other words of degree are relative modifiers intended to indicate permissible variation from the characteristic so modified (e.g., ±0.1%, ±0.5%, ±1.0%, ±2%, ±5%, ±10%, ±20%). It is not intended to be limited to the absolute value or characteristic which it modifies but rather possessing more of the physical or functional characteristic than its opposite, and preferably, approaching or approximating such a physical or functional characteristic.

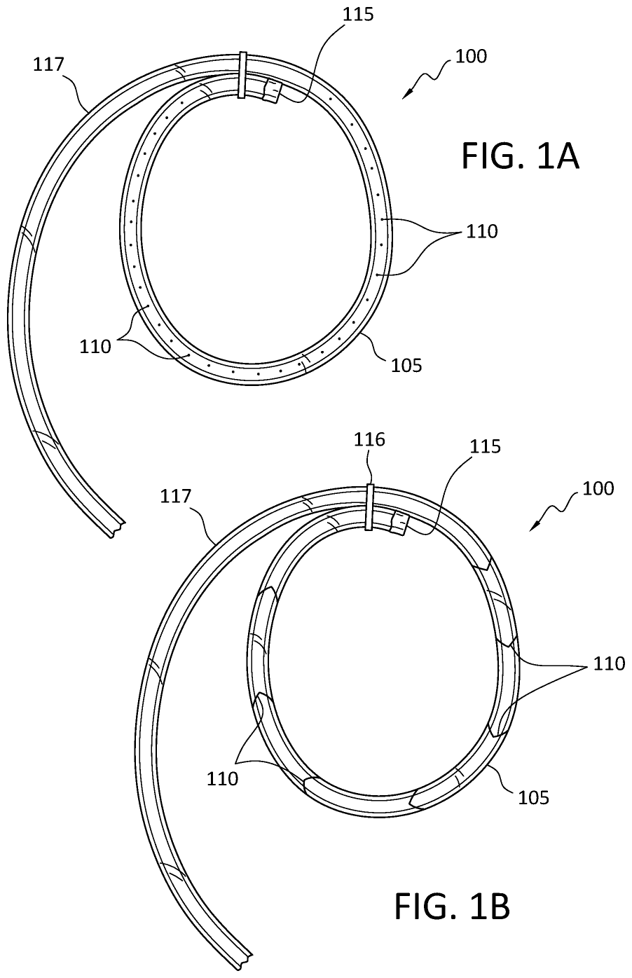

According to the present invention, as shown in FIGS. 1A-1D, an ocular injury treatment ring 100 comprises a substantially circular ring 105 configured to fit into the fornices of an eye. The circular ring has a shape and size that allows it to sit substantially in the fornices.

The substantially circular ring 105 comprises a plurality of fenestrations or holes 110. The plurality of fenestrations 110 may be located around the circumference of the circular ring 105, for example, along a length of material forming the circular ring (FIGS. 1A-1D). In embodiments, the circular ring may be made from any suitable medical grade material, including but not limited to, intravenous tubing or plastic, for example, intravenous or butterfly IV tubing (e.g., 0.5 mm or 0.8 mm tube diameter). In an embodiment, the substantially circular ring 105 may have a diameter of about 10 mm to about 40 mm, for example about 15 mm to about 30 mm.

In embodiments, each of the plurality of fenestrations 110 may be of a same size or a different size. The fenestrations are configured to create a circular and/or vortex pattern of a fluid as the fluid exits the fenestrations into the fornices and the rest of the eye. Each of the fenestrations may independently or together face outwardly from the ring or inwardly towards a center of the ring. In embodiments, each of the fenestrations may have an opening of about 0.1 mm to about 2 mm, for example, about 0.1 mm to about 0.5 mm. In embodiments, the number of fenestrations may be, but is not limited to, about 20 to about 80, for example about 20 to about 50. In a specific embodiment, the fenestrations may be made using a 16 to 20 gauge catheter needle, for example an 18 gauge catheter needle, inserted into tubing at an angle.

In a particular embodiment, each of the plurality of fenestrations may be angled with respect to a central longitudinal axis of the circular ring. Each fenestration may have an angle of about 10° to about 40°, for example about 20° to about 30°, with respect to a central longitudinal axis of the circular ring. The fenestrations may comprise an opening having any desired shape (e.g., circular, oval, square, rectangular, triangular), which may be non-angled or angled. In a particular embodiment, the fenestrations have an angled V-shaped opening (FIG. 1B).

In an embodiment, the plurality of fenestrations may comprise at least two sets of holes. Each set of holes may have the same size or a different size. The at least two sets of holes may comprise a first set of holes having a first size and a second set of holes having a second size larger than the first size, which may help provide an even distribution of liquid flow from the fenestrations. The first set of smaller holes may be located at or near an end of the circular ring where a liquid is initially pumped or inserted into the circular ring.

In an embodiment as shown in FIG. 1C, each set of holes may be positioned at a different location around a circumference of the circular ring (e.g., along a length of the tubing forming the circular ring) and around a circumference of the tubing forming the circular ring. For example, there may be four sets of holes, each set being located at successive quarters around the circumference of the circular ring and at successive quarter turns around the circumference of the tubing.

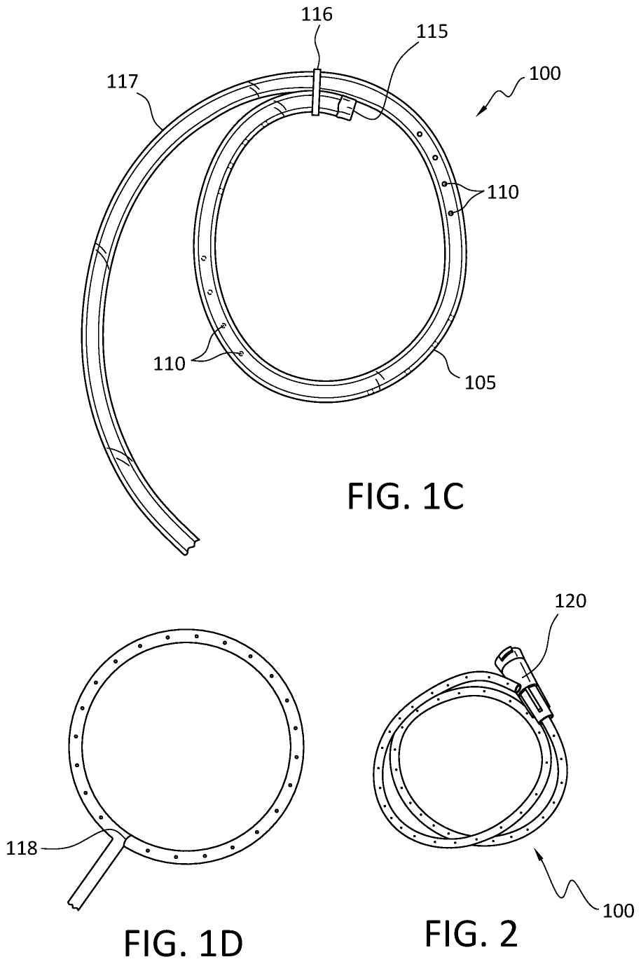

In embodiments, the circular ring 105 may have a cap 115 at a distal end (FIGS. 1A-1C). In a particular embodiment, the circular ring may be seared or singed closed at a distal end. At a proximal end, wherein a liquid enters the circular ring 105, the circular ring 105 may be integral with a section of tubing or plastic 117 that has no fenestrations and is connectable to a pump or to intravenous bag, for example, via connector 120, as shown in FIG. 2. In an embodiment, this section of tubing or plastic 117 may have at least one of an angle, bend, a tie 116 or a T-junction 118 (FIG. 1D) where it connects to the circular ring 105.

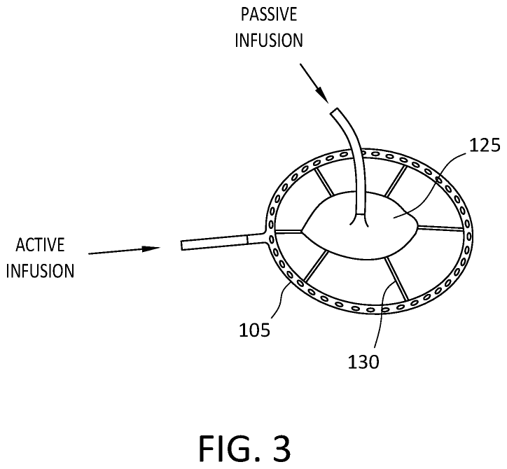

The circular ring may not have a corneal shield or dome or any ridges or fins attached thereto. However, in an embodiment as shown in FIG. 3, the circular ring 105 may comprise a central corneal shield 125 configured to vault over the cornea. The corneal shield 125 may have a passive infusion input for a liquid to be administered to the eye, for example, via an intravenous bag or drip; whereas the circular ring may have an active infusion input for a liquid, for example, via a pump. The circular ring may also have one or more flexible struts 130 extending from the corneal shield 125 to the circular ring 105. In an embodiment, there is a plurality of flexible struts 130 that allow or assist the ocular ring to expand into the fornices.

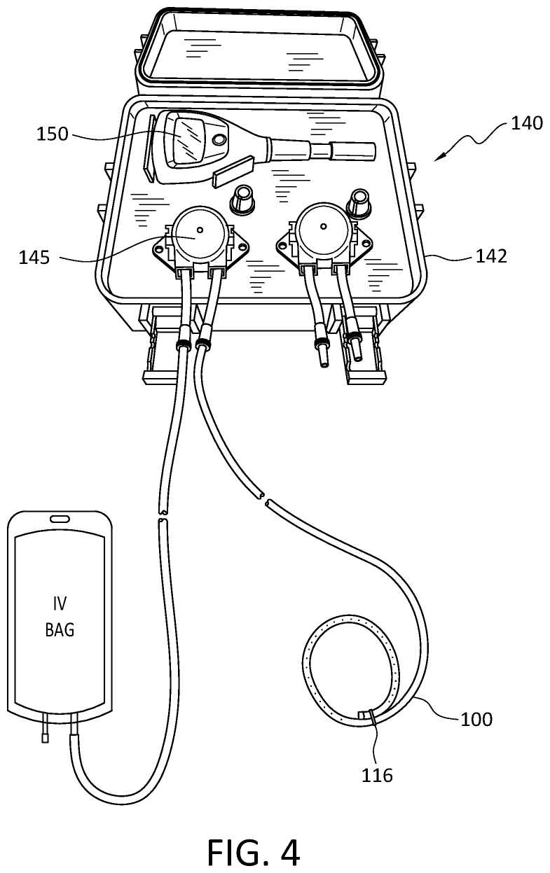

As shown in FIG. 4, the present invention is also directed to a kit 140 comprising a housing 142; at least one ocular chemical injury treatment ring 100 according to any embodiment of the present invention; at least one peristaltic and/or diaphragm pump 145 for providing a continuous flow of liquid to the at least one ocular ring; and at least one rechargeable battery or pack (e.g., a DC battery) for operating the at least one pump. In a specific embodiment, the at least one ocular ring may be in a separate sterilized package within the kit. In an embodiment, the pump may be connected to an IV bag via tubing and separately connected via IV tubing to at least one ocular chemical injury treatment ring 100.

A peristaltic pump may allow for a titratable and continuous flow of liquid to the circular ring. In a particular embodiment, the kit includes at least one 12V peristaltic configured to pump about 15 mL to about 500 mL of liquid per minute. The kit may also have a cord to allow for plugging into an outlet to provide power for the at least one pump.

The kit 140 may also include comprising a pH meter 150 having a single point configured to measure the pH of the eye. In embodiments, the pH meter is a unitary, one-piece body that is sterilizable and can be used on multiple patients. The PH meter does not have a separate sleeve and/or does not have any cracks, grooves, crevices, or divots.

The at least one of the pH meter or the at least one peristaltic and/or diaphragm pump may snap onto the housing. The kit has everything necessary for treating ocular chemical injuries, and the components are sterile and separate. Thus, the kit is ideal for treating eye injury prior to a hospital setting (e.g., emergency scene, battlefield, or the like). In a specific embodiment, the kit may be provided with two or more ocular rings, for example, a first ocular ring sized for children; a second ocular ring sized for adolescents; and a third ocular ring sized for adults.

According to the present invention, a method for treating an eye comprises inserting the ocular ring into an eye, such that the circumference of the circular ring is at least partially beneath the eyelids and substantially lies in or adjacent to the fornices; inserting or pumping a liquid into the circular ring and out of the plurality of fenestrations at a flow rate that creates a circular and/or vortex pattern in the fornices; and irrigating the fornices and cornea. Thus, chemical(s) and/or particulate matter is removed from the fornices. The ocular ring allows for substantially simultaneous corneal and fornix irrigation as fluid enters the eye in the fornices and exits between closed eyelids (the eye's palebral fissure). In an embodiment, the liquid to be supplied to the ocular ring may comprise at least one of saline solution, Ringer's lactate solution, or a medicine.

### INDUSTRIAL APPLICABILITY

The present invention is directed to an ocular chemical injury treatment ring and to methods of use thereof. In particular, the ocular chemical injury treatment ring is a fenestrated ring that is configured to fit into the fornices of the eye and may be used to treat chemical injuries to the eye.

Although the present invention has been described in terms of particular exemplary and alternative embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the exemplary and alternative embodiments described above can be configured without departing from the scope and spirit of the invention. Therefore, it is to be understood that, within the scope of the appended claims, the invention may be practiced other than as specifically described herein.

1. An ocular chemical injury treatment ring comprising:
a substantially circular ring comprising a medical grade material; and
a plurality of fenestrations around a circumference of the circular ring.

2. The ocular chemical injury treatment ring according to claim 1, wherein the circular ring comprises intravenous tubing or plastic.

3. The ocular chemical injury treatment ring according to claim 1, wherein the fenestrations are equally spaced about the circumference of the ring.

4. The ocular chemical injury treatment ring according to claim 1, wherein the fenestrations are of the same size.

5. The ocular chemical injury treatment ring according to claim 1, wherein the fenestrations are of a different size.

6. The ocular chemical injury treatment ring according to claim 1, wherein the plurality of fenestrations includes about 20 to about 50 fenestrations.

7. The ocular chemical injury treatment ring according to claim 1, wherein each of the plurality of fenestrations is angled with respect to a central longitudinal axis of the circular ring.

8. The ocular chemical injury treatment ring according to claim 1, wherein each fenestration comprises an angled V-shaped opening.

9. The ocular chemical injury treatment ring according to claim 1, wherein each of the fenestrations comprises an opening of about 0.1 mm to about 0.5 mm.

10. The ocular chemical injury treatment ring according to claim 1, wherein the circular ring has a diameter of about 10 mm to about 40 mm.

11. The ocular chemical injury treatment ring according to claim 1, wherein the plurality of fenestrations comprises a first set of holes having a first size and a second set of holes having a second size larger than said first size.

12. The ocular chemical injury treatment ring according to claim 1, comprising at least two sets of holes, wherein each set of holes is positioned at a different location around a circumference of the circular ring and around a circumference of tubing forming the circular ring.

13. The ocular chemical injury treatment ring according to claim 1, wherein the substantially circular ring has a cap at a distal end.

14. The ocular chemical injury treatment ring according to claim 1, wherein the substantially circular ring, at a proximal end, is integral with a section of tubing having no fenestrations and having a connector configured to attach to a pump or intravenous bag.

15. The ocular chemical injury treatment ring according to claim 1, having no corneal shield or dome.

16. The ocular chemical injury treatment ring according to claim 1, further comprising:
a central corneal shield; and
one or more flexible struts extending from the corneal shield to the circular ring.

17. A method for treating an eye, comprising:
inserting an ocular chemical injury treatment ring according to claim 1 into an eye of a patient, such that the circumference of the circular ring is at least partially beneath an eyelid of the patient and substantially in or adjacent to the fornices of the eye;
inserting or pumping a liquid into the circular ring and out of the plurality of fenestrations at a flow rate that creates a circular or vortex pattern in the fornices; and
irrigating the fornices and cornea of the eye.

18. A method according to claim 17, wherein the eye is treated for a chemical injury and said irrigating removes at least one of chemicals or particulate matter from the fornices.

19. A method according to claim 17, wherein the liquid comprises a medicine.

20. A kit, comprising:
a housing;
at least one ocular injury treatment ring according to claim 1;
at least one pump; and
a rechargeable battery.

21. The kit according to claim 20, further comprising: a pH meter, wherein said pH meter comprises a sterilizable unitary body having a single point configured to measure the pH of an eye and wherein the PH meter does not have any cracks, grooves, crevices, or divots.
